(19) United States
(12) Patent Application Publication
(10) Pub. No.: US 2008/0286243 A1
(43) Pub. Date: Nov. 20, 2008

(54) Method For Isolation of a Hair Follicle Stem Cell and a Composition For Hair Reproduction

(75) Inventors: Kyung Sun Kang, Seoul (KR); Jeong Chan Ra, Gyeonggi-do (KR)

Correspondence Address: INTELLECTUAL PROPERTY / TECHNOLOGY LAW, PO BOX 14329, RESEARCH TRIANGLE PARK, NC, 27709, US

(73) Assignees: SEOUL NATIONAL UNIVERSITY INDUSTRY FOUNDATION, Seoul (KR); RNL BIO CO., LTD., Seoul (KR)

(21) Appl. No.: 11/995,910

(22) PCT Filed: Jul. 4, 2007
(86) PCT No.: PCT/KR2007/003259

§ 371 Date: Jul. 3, 2008

(30) Foreign Application Priority Data
Jul. 5, 2006 (KR) 10-2006-0062807

(51) Int. Cl. A61K 35/12; C12N 5/02; A61P 17/14

(52) U.S. Cl. 424/93.7; 435/392; 435/384

(57) ABSTRACT

The present invention relates to a method for isolating hair follicle stem cells and a composition for inducing hair growth. More specifically, relates to a method for isolating hair follicle stem cells showing a positive immunological response to CD34, by chemically degrading hair follicle-containing scalp tissue and then culturing the degraded tissue in a serum-containing medium and a serum-free medium, as well as a composition for inducing hair growth, which contains, as an active ingredient, CD34-positive hair follicle stem cells isolated by the method. The hair follicle-derived stem cells, which are obtained according to the disclosed method, are classified as autologous adult stem cells, have self-renewal capability, the ability to differentiate into adult hair follicle cells and the ability to induce hair growth, and can be used as a novel cell therapeutic agent against hair loss. In addition, the present invention relates to a method for culturing hair follicle cells, which has high yield compared to that of the prior art, as well as a method for identifying hair follicle stem cells.

### TECHNICAL FIELD

The present invention relates to a method for isolating hair follicle stem cells and a composition for inducing hair growth, and more particularly to a method of isolating hair follicle stem cells showing a positive immunological response to CD34, by chemically degrading hair follicle-containing scalp tissue and then culturing the degraded tissue in each of serum-containing medium and serum-free medium, as well as a composition for the induction of hair growth, which contains, CD34-positive hair follicle stem cells isolated by the method, as an active ingredient.

### BACKGROUND ART

Recently, with an increasing interest in beauty, an interest in the treatment of alopecia has also increased. Alopecia refers to hair loss in areas of skin that normally have hair. Alopecia can be divided into scarring alopecia where the skin scars, and non-scarring alopecia where only hair falls out. In scarring alopecia, hair follicles are permanently destroyed and hair never regrows. Hair is made in hair follicles, and each follicle undergoes repeated cycles of active growth and rest and has about 10-20 hair follicle growth cycles in a person's lifetime (Cotsarelis, G. et al., Cell, 61(7):1329, 1990). Generally, 85-95% of hairs are in the anagen stage, and the number of anagen-phase follicles decreases as a person grows old. Thus, 10-15% of hairs are in the catagen phase or telogen phase, and an average of about 50-60 hairs normally fall out daily. A hair loss of more than 100 hairs a day can lead to alopecia.

Various methods for the treatment of hair loss have been suggested, and among them, a surgical method, which is currently most frequently used, is autologous hair transplantation, in which follicles are removed from a nonbalding region of the scalp and transplanted to the balding region. However, this method has a problem in that the transplanted hair often looks floppy.

With respect to drug therapy, only two drugs, Propecia for oral administration and Minoxidil for skin application, have been approved for use by the US FDA. Propecia shows therapeutic effects during the administration period, but if the administration thereof is stopped, hair loss develops again like before the drug is administered (Bouhanna, P., Dermatol. Surg., 29(11):1130, 2003; Thiboutot, D., Arch. Dermatol., 135(11):1417, 1999). Meanwhile, Minoxidil is a lair loss treatment drug which can be used for males and females (Bouhanna, P., Dermatol. Surg., 29(11):1130, 2003; Messenger, A. G. & Rundegren, J., Br. J. Dermatol., 150(2):186, 2004). However, it has been reported that such drugs have a lot of side effects, including sexual dysfunction (Messenger, A. G. & Rundegren, J., Br. J. Dermatol., 150(2):186, 2004).

Meanwhile, gene therapeutic methods have recently received attention. Since genes, which is involved in diseases causing general hair loss, were found and reported (Ahmad, W. et al., Science, 279(5351):720, 1998), therapeutic methods of delivering target DNA codes directly to hair follicles using such gene structures or inhibiting the expression of the genes have been actively developed. However, the efficiency, therapeutic cost, safety, effects on future generations, etc. of such therapeutic methods are not yet sufficiently studied, and thus, even if genes, which are involved in hair loss, are found, a considerable period of time will be required to achieve the practical application of therapeutic methods which employ the genes.

Also, U.S. Pat. No. 6,399,057 discloses a method for the regeneration of hair, comprising removing hair in the anagen phase from donor regions by plucking so that a bulb is still attached to the hair removed, culturing the hair follicle cells of the removed hair, and then implanting the cultured cells into pores of receptor regions. However, this method is considered to be insignificant.

Meanwhile, adult stem cells have received a great deal of attention as cell therapeutic agents against many diseases. As used herein, the term “adult stem cells” refers to cells from all adult organs, which exhibit self-renewal, self-maintenance and multipotency. Up to date, the characteristics of adult stem cells and operation thereof have been applied in a wide range of clinical areas. For example, the identification of corneal epithelial stem cells inside the eyeball led to the development of new techniques for corneal transplantation (Cotsarelis, G. et al., Cell, 57:201, 1989; Tsai, R. J. et al., N. Engl. J. Med., 343:86, 2000) and resulted in autologous stem cell transplantation and gene therapy due to the characteristics of hematopoietic stem cells (Bernstein, I. D. et al., Blood Cells, 20:15, 1994). Also, the concept of epidermal stem cells has been studied 30 years ago (Potten, C. S., Cell Tiss. Kinet., 7:77, 1974). More recent in vitro studies based on cell culture studies (Barrandon, Y. & Green, H., Proc. Natl. Acad. Sci. USA, 84:2303, 1987; Rochat, A. et al., Cell, 76:1063, 1994) and in vivo studies in mice have suggested a somewhat more complex organization and distribution of stem cells in skin, with stem-like cells implicated at specific locations in the interfollicular epidermis, in the upper regions of the outer root sheath of the hair follicle (the so-called bulge region), and in the germinal matrix of growing hair follicles.

The interrelationship between these three separate skin stem cell compartments remains obscure, although it can be hypothesized that the bulge region stem cells represent the most potent reserve population of ultimate stem cells. Also, it has been consistently reported that follicle stem cells in the bulge region express CD34 cell surface protein in rats (Trempus, C. S. et al., J. Invest. Dermatol., 120(4):501, 2003). However, it has been reported that the cells of the bulge region in human do not express CD34. Also, whether such CD34-negative cells cause hair growth in hair follicles is not yet found. Furthermore, a culture method for such follicle stem cells was not clearly established, and a marker for the stem cells was unclear. Moreover, although it is known that follicle stem cells are present in some hair follicles, a large amount of stem cells are required for the practical treatment of human baldness, but technology of proliferating isolated stem cells as much as they can be clinically applied is still unsatisfactory. In addition, a marker protein for the stem cells is not yet clearly identified, and thus a method for treating hair loss using the stem cells is still unsatisfactory.

Accordingly, the present inventors have made many efforts to develop a method for the culture of hair follicle cells and a method for the identification of hair follicle stem cells and to use stem cells for the treatment of alopecia, atrichia and the like in beauty and medical fields. As a result, the present inventors have isolated hair follicle stem cells by culturing hair follicle-containing scalp tissue and found that it is a more efficient method to obtain a high yield of hair follicle stem cells, compared to the prior art, and that a composition containing the isolated hair follicle stem cells is effective in the induction of hair growth, that is, the treatment of alopecia, thereby completing the present invention.

### SUMMARY OF THE INVENTION

It is an object of the present invention to provide a method for isolating hair follicle stem cells by culturing hair follicle-containing scalp tissue.

Another object of the present invention is to provide a composition for the induction of hair growth, which contains the hair follicle stem cells obtained using said method, as an active ingredient.

To achieve the above objects, the present invention provides a method for isolating hair follicle stem cells, the method comprising the steps of: (a) cutting hair follicle-containing scalp tissue into fine pieces and chemically degrading the cut tissue; (b) collecting the chemically degraded tissue and culturing the collected tissue in a medium containing 1-30 vol % of serum, in an incubator; (c) replacing the medium with a serum-free medium, when the tissue adheres to the incubator, and then re-culturing the tissue; and (d) collecting the cultured and proliferated scalp cells from the re-cultured tissue, and isolating hair follicle stem cells showing a positive immunological response to CD34, from the collected cells.

The present invention also provides a composition for the induction of hair growth, which contains said isolated CD34-positive hair follicle stem cells as an active ingredient.

Other features and aspects of the present invention will be apparent from the following detailed description and the appended claims.

### BRIEF DESCRIPTION OF DRAWINGS

FIG. 1 shows photographs illustrating the configuration of cells, which started to adhere to a cell culture dish when cultured in a defined keratinocyte serum-free medium containing no normocin. The two photographs show different regions of the same flask at 100× magnification.

FIG. 2 shows photographs illustrating the configuration of cells after the first passage culture, which started to adhere to a cell culture dish when cultured in a defined keratinocyte serum-free medium containing no normocin. The two photographs show different regions of the same flask at 100× magnification.

FIG. 3 shows photographs illustrating the configuration of cells, which did not adhere to a dish at the initial stage when cultured in a defined keratinocyte serum-free medium containing no normocin, but adhered to the dish after transferred into a flask. The two photographs show different regions of the same flask at 100× magnification.

FIG. 4 shows a growth curve of hair follicle cells, plotted by collecting cells at 3 days after the start of culture, calculating the cell number every day for 1-8 days of subculture of the cells, and calculating the mean and standard deviation values of the calculated cell number each day.

FIG. 5 schematically shows a method for isolating CD34-positive cells using MACS (magnetic cell sorting).

FIG. 6 shows an MACS system used in the isolation of CD34-positive cells.

FIG. 7 shows the immunological characteristics of hair follicle stem cells, measured using a FACS (fluorescence activated cell sorting) technique.

FIG. 8 shows photographs of a control mouse group (A) without hair follicle stem cells administration, and a mouse group (B) with hair follicle stem cells administration.

FIG. 9 shows the results of H&E staining of scalp tissues of a control mouse group (A) without hair follicle stem cells administration, and a mouse group (B) with hair follicle stem cells administration.

FIG. 10 shows the results of in situ hybridization conducted using a human-specific probe on groups (A and B) with hair follicle stem cells administration, and groups (C and D) without hair follicle stem cells administration.

### DETAILED DESCRIPTION OF THE INVENTION, AND PREFERRED EMBODIMENTS

In one aspect, the present invention relates to a method for isolating hair follicle stem cells from hair follicle-containing scalp tissue.

In one embodiment of the present invention, hair follicle stem cells can be isolated through the following steps: (a) cutting hair follicle-containing scalp tissue into fine pieces and chemically degrading the cut tissue; (b) collecting the chemically degraded tissue and culturing the collected tissue in a medium containing 1-30 vol % of serum, in an incubator; (c) replacing the medium with a serum-free medium when the tissue adheres to the incubator, and then re-culturing the tissue; and (d) collecting the cultured and proliferated scalp cells from the re-cultured tissue, and isolating hair follicle stem cells showing a positive immunological response to CD34, from the collected cells.

More specifically, the chemical degradation of step (a) may comprise the sequential sub-steps of: (i) degrading the tissue in a medium, which contains a protein complex comprising DNase and protease, and a dispase; (ii) degrading the tissue in a collagenase-containing medium.

In the present invention, the medium containing 1-30 vol % of serum, used in the step (b), is preferably a mixed medium of M199 and F12 (1:1 v/v), added with 0.1˜1.0 μg/ml of insulin, 0.1˜1.0 μg/ml of transferrin, 50-150 units of penicillin, 0.05-0.15 mg/ml of streptomycin, 0.1-0.5 μg/ml of neomycin, 1-100 ng/ml of rEGF (epidermal growth factor), 1-100 ng/ml of bFGF (basic fibroblast growth factor), 10-200 μg/ml of normocin, 0.01-0.3 ml/ml (1-30 vol %) of fetal bovine serum and 0.1-10 mM of N-acetyl-L-cystein.

Also, in the present invention, the serum-free medium in the (c) is preferably a serum-free medium containing no normocin. Specifically, the serum-free medium containing no normocin is preferably a serum-free keratinocyte medium containing 0.1-10 mM ascorbic acid.

Moreover, the step (c) in the present invention may further comprise, culturing the tissue in a serum-free medium containing normocin, before culturing the tissue in the serum-free medium containing no normocin. Herein, the serum-free medium containing normocin is preferably a mixed medium of M199 and F12 (1:1 v/v), added with 0.1-1.0 μg/ml of insulin, 0.1-1.0 μg/ml of transferrin, 50-150 units of penicillin, 0.05-0.15 mg/ml of streptomycin, 0.1-0.5 μg/ml of neomycin, 1-100 ng/ml of rEGF (epidermal growth factor), 1-100 ng/ml of bFGF (basic fibroblast growth factor), 10-200 μg/ml of normocin and 0.1-10 mM of N-acetyl-L-cystein.

Hair follicle stem cells that can be used in the present invention can be isolated from the scalp tissue of all kinds of mammals, in which the scalp tissue should contain hair follicles. If human scalp tissue is used, it is preferably hair follicle-containing scalp tissue resulting from surgical hair transplantation. In surgical hair transplantation, the scalp is removed from the side and back (donor regions) of the head, in which alopecia does not develop in a lifetime, and then the removed hair-baring scalp is separated into small grafts (1-4 hair follicles) or large grafts (3-6 hair follicles) using a magnifying glass, and the separated grafts are transplanted into balding regions and the regions between hairs having a reduced diameter (receptor regions). The scalp tissue that remains after the transplantation is used in the present invention.

In order to isolate hair follicle stem cells from scalp tissue, hair follicle-containing scalp tissue is first cut into fine pieces, which are then chemically degraded. In the chemical degradation process, first step chemical degradation is performed in a medium, comprising dispase and a protein complex containing DNase and protease. The medium is preferably a DMEM medium containing 0.01-0.3 ml/ml (1-30 vol %) of fetal bovine serum, 10-200 μg/ml of normocin, 50-150 units of penicillin, 0.05-0.15 mg/ml of streptomycin, 0.1-0.5 μg/ml of neomycin, added with 0.1-2 mg/ml of dispase and 0.01-0.1 ml/ml (1-10 vol %) of a protein complex containing DNase and protease, but the scope of the present invention is not limited thereto. Herein, the protein complex containing DNase and protease is commercially available, and for example, accumax (Chemicon cat# SCR006) may be used as the protein complex, but the scope of the present invention is not limited thereto.

Thereafter, second step chemical degradation is performed in a collagenase-containing medium. Herein, the collagenase-containing medium is preferably a DMEM medium containing fetal bovine serum, normocin, penicillin, streptomycin, added with collagenase type IA. The DMEM medium preferably contains 0.01-0.3 ml/ml (1-30 vol %) of fetal bovine serum, 10-200 ug/ml of normocin, 50-150 units of penicillin, 0.05-0.15 mg/ml of streptomycin, 0.1-0.5 μg/ml of neomycin, but the scope of the present invention is not limited thereto and the collagenase type IA is preferably added to the DMEM medium in an amount of 0.1˜10 mg/ml. Also, the chemical degradation (first step and second step degradation) of the finely cut tissue is preferably carried out in a gravity convection incubator at 50-200 n/min and 30-40□ for 0.5-24 hours.

Then, the chemically degraded tissues (scalp cells) are collected, and cultured in a medium, which contains serum, preferably 1-30 vol % of fetal bovine serum. Herein, the medium is preferably a mixed medium of M199/F12. Then, when the tissues adhere to the flask, the medium is replaced with a serum-free medium. Generally, after the tissues are cultured for 3 days to 1 month, the medium is replaced with a serum-free medium containing no normocin.

The mixed medium of M199/F12, which is used in the initial stage of the culture of the scalp cell, is preferably a medium obtained by mixing M199 with F12 at a volume ratio of 1:1 and adding thereto 0.1-1.0 μg/ml of insulin, 0.1-1.0 μg/ml of transferrin, 50-150 units of penicillin, 0.05-0.15 mg/ml of streptomycin, 0.1-0.5 μg/ml of neomycin, 1-100 ng/ml of rEGF (epidermal growth factor), 1-100 ng/ml of bFGF (basic fibroblast growth factor), 10-200 μg/ml of normocin, 0.01-0.3 ml/ml (1-30 vol %) of fetal bovine serum, and 0.1-10 mM of N-acetyl-L-cystein. And the medium that is used after the cell adhesion is preferably a commercially available serum-free keratinocyte medium containing 0.1-10 mM of ascorbic acid, but the scope of the present invention is not limited thereto. Herein, the serum-free keratinocyte medium is commercially available and may be, for example, a defined keratinocyte serum-free medium (Gibco cat# 10785-012), but the scope of the present invention is not limited thereto.

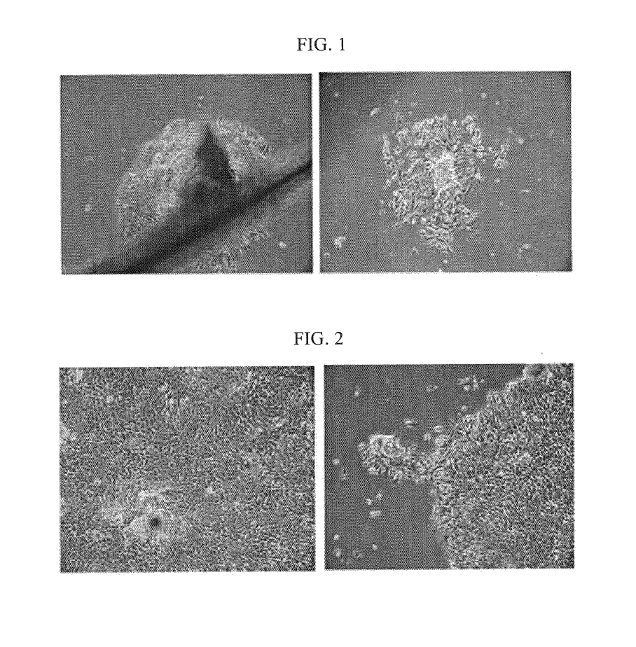

When the cells start to adhere to a dish about 2 days after replacement of the medium (see FIG. 1), the floating tissues and cells are transferred into a flask having a larger size. When the cells that started to adhere are treated with trypsin and subcultured for about 5-15 days, the cells are grown to a confluency of about 70-90% in the flask. From this point of time, the medium is replaced with the same fresh medium, after cells are washed two times with phosphate buffered saline at an interval of 2-3 days.

Thereafter, the cultured scalp cells are collected, and among them, CD34-positive cells are isolated, thereby isolating hair follicle stem cells. Herein, the method of isolating the CD4-positive cells from the cultured scalp cells can be performed using MACS (magnetic cell sorting), which is conventionally known in the art. The MACS system can be commercially available from Miltenyi Biotec Inc.

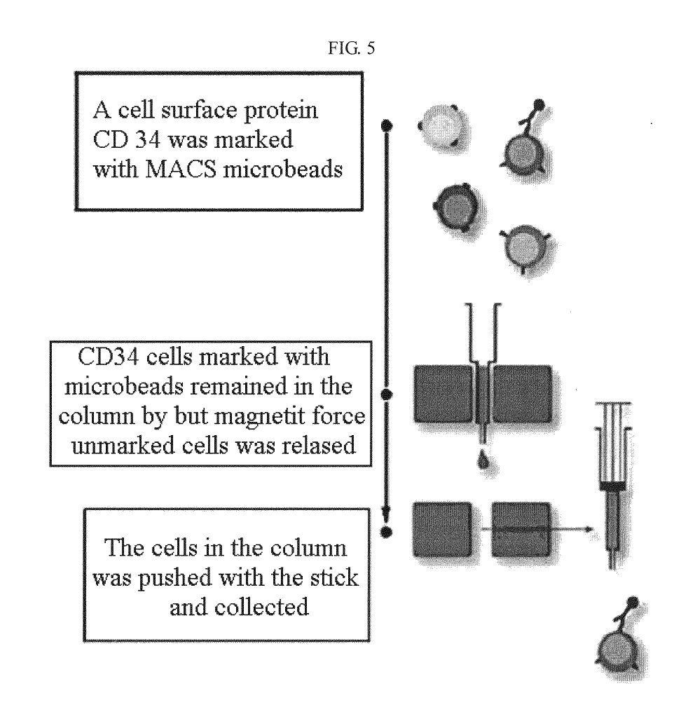

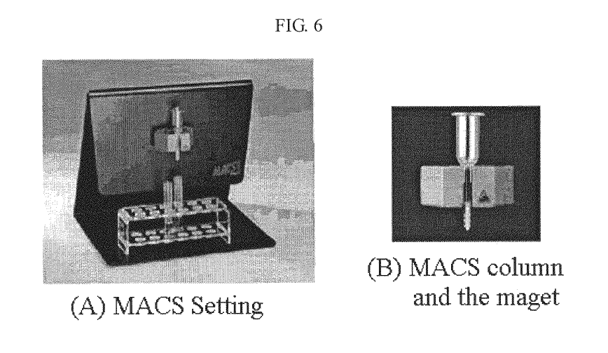

The method of isolating the CD34-positive cells using MACS is schematically shown in FIG. 5.

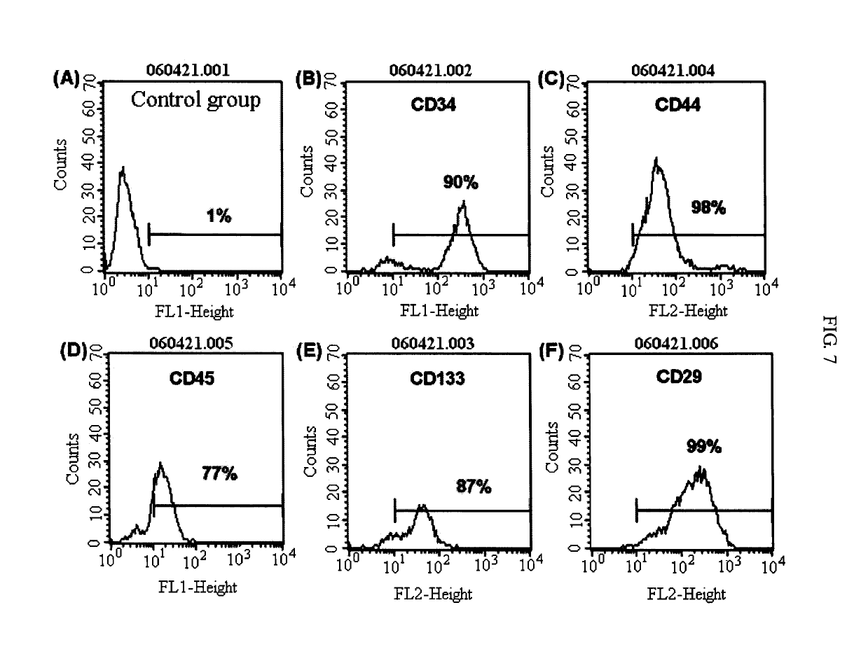

The collected scalp cells are singled out, and MACS buffer, a blocking reagent and microbeads are sequentially added thereto. Herein, the amounts of the reagents are 150-1000 μl for the MACS buffer, 50-500 μl for the blocking reagent, and 50-500 μl for the microbeads, and the culture time of the cells is 30 minutes to 4 hours. In all the processes of handing the blocking reagent and the microbeads, a fluorescent lamp should be turned off. The tube containing the mixture is wrapped with an aluminum foil, and the mixture is cultured at 4□ for 30 minutes to 4 hours. Then, the aluminum foil is removed, and MACS buffer having a volume about 10 times that of the mixture is added to the tube and is sufficiently stirred with a pipette. Then, the mixture solution is centrifuged at 1200 rpm for 5-6 minutes, and the supernatant is removed. Then, MACS buffer is added to the remaining pellets in an amount corresponding to the number of the cells, and the pellets are suspended using a pipette. Herein, the amount of the MACS buffer is 500 μl. The tube containing the cells mixed with the MACS buffer is put on ice, the MACS system is set, and a MACS column and a magnet are set (FIG. 6). Briefly, the setting process is performed in the following manner. Conical tubes marked with ‘CD+’ and ‘CD−’, respectively, are prepared. A black support (steel) is placed at a suitable location, and a green magnet is attached to the support at a suitable height. The MACS column is inserted into a groove in the magnet with care in order for the column not to be contaminated.

Among the prepared tubes, the tube marked with ‘CD34−’ is placed such that it can receive cells dropping from the column. After completion of the setting process, 150˜1000 μl of MACS buffer is allowed to run through the column. After all the MACS buffer almost run down, 500 μl (2×10⁸ cells) of the cell mixture kept on ice is put in the column. The cell mixture slowly runs down along the column, and cells that fall down from the column are CD34− cells. CD34-positive cells remain in the black portion in the middle of the column. After the mixture solution containing the cells in an amount corresponding to the receiving capacity of the column is allowed to run through the column, about 500 μl of MACS buffer is loaded into the column to allow the CD34− cells remaining in the column to run down. Then, the MACS column is taken out from the magnet, and a stick provided together with the column is inserted into the upper portion of the column and pushed such that the column is placed in the tube marked with ‘CD34+’. Medium is poured into the column and pushed with the stick, and the medium comes out through the column is received, thus obtaining CD34-positive cells contained in the received medium.

The immunophenotype antigen of the hair follicle stem cells, which are isolated according to the present invention, displays CD34 positivity and also shows one or more immunological characteristics selected from among CD44 positivity, CD45 positivity, CD133 positivity and CD29 positivity.

When the isolated hair follicle stem cells that are CD34-positive cells are injected subcutaneously into scalp tissue, they will differentiate into hair follicle cells. Furthermore, the differentiated hair follicle cells have the ability to grow hair continuously, that is, the ability to induce hair growth. As used herein, the term “hair growth induction” refers to the ability to induce hair growth by forming hair follicles in hair loss regions or hairless regions. Accordingly, the isolated hair follicle stem cells can be used for the treatment of balding.

Thus, in another aspect, the present invention provides a composition for the induction of hair growth, which contains the CD34-positive hair follicle stem cells isolated according to the above-described method, as an active ingredient.

The dose of the hair follicle stem cells as an active ingredient, which are contained in the composition, is more than 1×10³ cells, preferably 1×10³ to 1×10⁹ cells, and more preferably 1×10³ to 1×10¹² cells, but the scope of the present invention is not limited thereto. The composition is preferably administered by subcutaneous injection and may be administered in a single dose or multiple doses. However, it is to be understood that the actual dose should be determined depending on various factors, including patients' age, sex, weight and disease severity, and thus, the above-specified dose should not be construed as limiting the scope of the present invention in any way. Also, the hair growth-inducing composition of the present invention can be prepared by mixing the isolated hair follicle stem cells with a carrier, an excipient or a diluent. For example, the hair follicle stem cells may be mixed with sterilized physiological saline.

Moreover, scalp tissue, to which the hair follicle stem cells are administered, may be the scalp tissue of a subject, from which the hair follicle stem cells were originally derived. Alternatively, it may also be the scalp tissue of other subjects. Preferably, it is the scalp tissue of a subject, from which the hair follicle stem cells were originally derived. Furthermore, the scalp tissue is preferably that of mammals. Mammals that can be used in the present invention include human, rats, pigs, cattle, dogs, cats, etc.

### EXAMPLES

Hereinafter, the present invention will be described in further detail with reference to examples. It will be apparent to one skilled in the art that these examples are for illustrative purpose only and are not construed to limit the scope of the present invention.

### Example 1

### Isolation of Hair Follicle Stem Cells

### (1) Culture of Scalp Cells

From scalp including subcutaneous tissue, collected from the back of the head of a surgical hair transplant patient, scalp tissue remaining after surgical transplantation was obtained. The obtained hair follicle-containing scalp was placed in a DMEM medium containing 10 vol % of 0.1 ml/ml of fetal bovine serum, 100 μg/ml of normocin, 100 units of penicillin, 0.1 mg/ml of streptomycin and 0.25 μg/ml of neomycin. The scalp was taken out using sterilized forceps, placed in a Petri dish and cut into fine pieces using a blade inserted into a scalpel. The cut tissue was added to a DMEM medium containing 0.1 ml/ml (10 vol %) of fetal bovine serum, 100 units of penicillin, 0.1 mg/ml of streptomycin, 0.25 μg/ml of neomycin, 100 μg/ml of normocin, added with 2 vol % (0.02 ml/ml) of accumax (Chemicon cat# SCR006) and 0.4 mg/ml of dispase, in a Petri dish, and was transferred into a flask. The tissue contained in the flask was subjected to first step chemical degradation in a gravity convection incubator at 130 m/min and 37□ for 30 minutes.

Then, the chemically degraded tissue was collected by centrifugation and washed 3 times with phosphate buffered saline. The washed tissue was subjected to second-step chemical degradation in a collagenase-containing medium (a DMEM medium containing 10 vol % of fetal bovine serum, 100 units of penicillin, 0.1 mg/ml of streptomycin, 0.25 μg/ml of neomycin, 100 μg/ml of normocin added with 2 mg/ml of collagenase type IA) in a gravity convection incubator at 130 m/min and 37□ for 30 minutes. The chemically degraded tissue was collected by centrifugation and washed 3 times with phosphate buffered saline. Then, the collected tissues were cultured in a T-25 flask containing a serum-containing M199/F12 medium (obtained by mixing M199 with F12 at a volume ratio of 1:1 and adding thereto 0.62 μg/ml of insulin, 0.62 μg/ml of transferrin, 100 units of penicillin, 0.1 mg/ml of streptomycin, 0.25 μg/ml of neomycin, 10 ng/ml of rEGF, 10 ng/ml of bFGF, 100 μg/ml of normosin, 0.1 ml/ml (10 vol %) of fetal bovine serum and 1 mM of N-acetyl-L-cystein). When the tissue started to adhere to a dish (after about 3 days), the medium was replaced with a fetal bovine serum-free M199/F12 medium (obtained by eliminating only fetal bovine serum from said serum-containing M199/F12 medium).

After one week, the medium was replaced with a normocin-free, defined keratinocyte serum-free medium (Gibco cat# 10785-012) supplemented with 0.2 mM of ascorbic acid, and after 2 days, the configuration of cells, which started to adhere to the dish, was photographed (see FIG. 1). Two photographs in FIG. 1 show different regions of the same flask at 100× magnification.

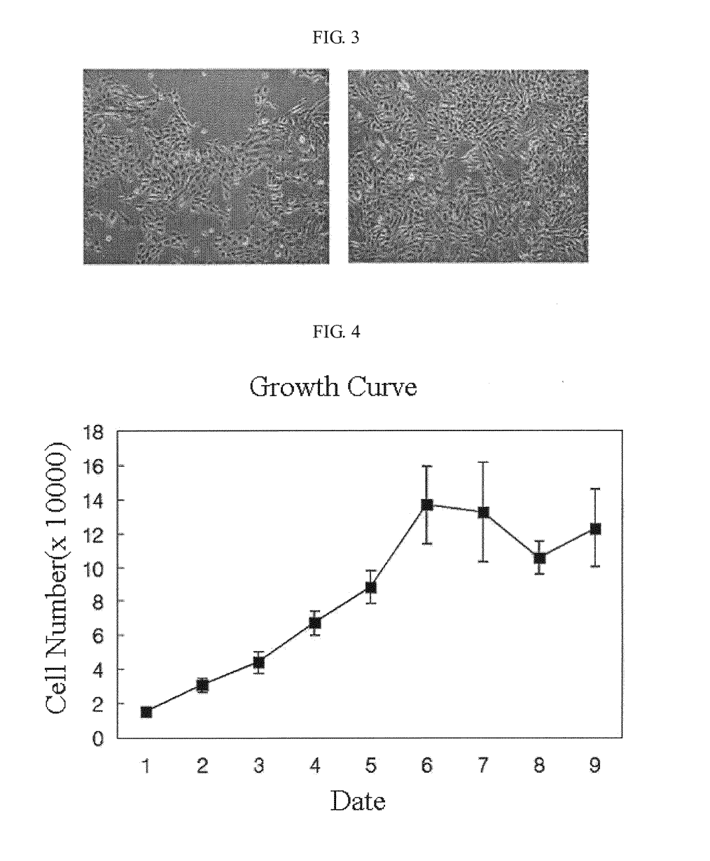

When the cells started to adhere to the dish 2 days after replacement of the medium (FIG. 1), the floating tissue and cells were transferred into a T-75 flask. The cells that started to adhere were treated with trypsin, and then subcultured for about 5-7 days. As a result, the cells were grown to a confluency of about 70-90% in the flask. The configuration of the cells after the first passage culture is shown in FIG. 2. Two photographs in FIG. 2 show different regions of the same flask at 100× magnification. From this point of time, the medium in the flask was replaced with the same fresh medium at an interval of 2-3 days, after the culture medium was washed two times with phosphate buffered saline. At 7 days after the cells were transferred to the T-75 flask, the cells attached to the T-25 flask were treated with trypsin and subcultured in a T-75 flask (cell number 1), and then cells, which did not adhere to the T-75 flask, to which the cells were transferred for the first time, were transferred again (cells attached to the dish: cell number 2; and floating cells: cell number 3). When the cells were grown to a confluency of about 90% in the flask of cell number 1 (FIG. 2) and the flask of cell number 2 (FIG. 3), all the cells were collected.

The cells, which floated at the initial culture stage in the T-25 flask, were transferred into a T-75 flask. The cells at 2 weeks of culture after starting to adhere to the dish in the T-75 flask were grown to a confluency of about 90% in the flask. The configuration of the cells, which did not adhere to the dish at the initial stage of culture, but adhered to the dish after transferred into the T-75 flask, is shown in FIG. 3. Two photographs in FIG. 3 show different regions of the same flask at 100× magnification.

### (2) Growth Curve for Hair Follicle Cells

At 3 days after the start of culture, the cells were treated with trypsin, and then seeded into four 6-well plates at a density of 1×10⁴ cells/well. Then, the cells were cultured for 8 days, while the number of cells in three wells was calculated every day. The mean and standard deviation of the calculated values were calculated, and a growth curve was plotted on the basis of the calculation results (see FIG. 4).

(3) Isolation of Stem Cells from Scalp Cells

Using a commercially available MACS (Magnetic Cell Sorting) system (Miltenyi Biotec Inc.), CD34-positive cells were isolated. A method of isolating the CD34-positive cells using MACS is schematically shown in FIG. 5.

Specifically, the scalp cells, cultured according to the above-mentioned method, were treated with trypsin and centrifuged at about 1200 rpm for 5 min to remove the medium while leaving cell pellets. A suitable amount (about 10 ml) of medium was placed in a tube containing the cell pellets, and the pellets were re-suspended in the medium using a pipette.

The suspension was centrifuged to remove the medium while leaving the cell pellets. The pipetting operation was preferably carried out after the pellets in the tube are singled out by tapping the tube with the finger. MACS buffer, a blocking reagent and microbeads were sequentially added into the tube. Herein, the amounts of materials added were 150-300 μl for the MACS buffer, 50-100 μl for the blocking reagent and 50-100 μl for the microbeads, and the culture time was in the range from 30 minutes to 4 hours. In the process of handing the blocking reagent and the microbeads, a fluorescent lamp was turned off. The tube containing the mixture solution was wrapped with an aluminum foil, and the mixture in the tube was cultured at 4□ for 30 minutes to 4 hours. Then, the aluminum foil was removed and MACS buffer having a volume about 10 times that of the mixture was added to the tube. Thereafter, the solution was sufficiently stirred with a pipette, and then centrifuged at 1200 rpm for 5-6 minutes, and the supernatant was removed. Then, MACS buffer was added to the remaining cell pellets in an amount corresponding to the number of the cells, and the pellets were suspended with a pipette. Herein, the amount of MACS buffer added was 500 μl. The tube containing the cells mixed with MACS buffer was placed on ice, and a column and a MACS kit were set. It is possible to attach a triangular green magnet to any suitable location of a black support.

FIG. 6 shows the MACS system. When cells marked with CD34 microbeads were prepared, the MACS system was set (FIG. 6(A)), and the MACS column and the magnet were suitably set (FIG. 6(B)).

Briefly, the setting process was performed in the following manner. 50 ml tubes marked with ‘CD+’ and ‘CD−’, respectively, were prepared. The black support (steel) was placed at a suitable location, and the green magnet was attached to the support at a suitable height. The column was inserted into a groove of the magnet with care in order for the column not to be contaminated.

Among the prepared tubes, the tube marked with ‘CD34−’ was placed such that it could receive cells falling down from the column. After completion of the setting process, 200-500 μl of MACS buffer was allowed to run through the column. When almost all the MACS buffer ran down, 500 μl (2×10⁸ cells) of the cell mixture solution, which has been kept on ice, was placed in the column. The cell mixture solution slowly flowed down along the column, and cells falling down from the column were CD34− cells. CD34+ cells remained in the black portion in the middle of the MACS column. When the mixture solution containing the cells in an amount corresponding to the receiving capacity of the column was allowed to run through the column, about 500 μl of MACS buffer was loaded into the column to allow the CD34− cells remaining in the column to run down. Then, the MACS column was taken out from the magnet, and a stick provided together with the column was inserted into the upper portion of the column and pushed to place the column in the tube marked with ‘CD34+’ (just like the use of a syringe). About 200-300 μl of medium (corresponding to the target cells) was poured into the column and pushed with the stick, and the medium came out through the column was received in the tube marked with CD34+. 10 ml of medium was placed in the tube containing the CD34-positive cells. At this time, the medium was sprayed to the wall of the tube, such that cells, which splashed when the column was pushed with the stick, were all washed down, and then CD34-positive cells were collected. The number of the collected CD34-positive cells was about 1×10⁷-2×10⁷, and the cells were centrifuged at 1500 rpm for 5-6 minutes, and the supernatant was removed. The CD34-positive cells contained in the tube were re-suspended in 100 μl of sterilized physiological saline and transferred into a 500 μl syringe.

Meanwhile, the yield of the collected CD34-positive cells isolated using MACS was calculated according to the following equation:

$$Yield ( \% ) = \frac{Number of CD 34 + cells}{Total number of cells before MACS} \times 100$$

The measurement results are shown in Table 1 below.

TABLE 1

| | Total number | Number of | |
|---|---|---|---|
| | of cells | CD34+ cells | Yield of |
| Test # | before MACS | after MACS | CD34+ cells (%) |
| 1 | 3.00 × 10⁷ | 1.28 × 10⁷ | 42.6666667 |
| 2 | 4.57 × 10⁷ | 1.19 × 10⁷ | 26.0393873 |
| 3 | 6.80 × 10⁷ | 1.43 × 10⁷ | 21.0294118 |
| 4 | 7.66 × 10⁷ | 1.81 × 10⁷ | 23.6292428 |
| 5 | 6.10 × 10⁷ | 2.88 × 10⁷ | 47.2131148 |
| 6 | 4.77 × 10⁷ | 1.14 × 10⁷ | 23.8993711 |
| 7 | 3.77 × 10⁷ | 1.64 × 10⁷ | 43.5013263 |
| 8 | 3.55 × 10⁷ | 2.04 × 10⁷ | 57.4647887 |
| 9 | 3.30 × 10⁷ | 1.15 × 10⁷ | 34.8484848 |
| 10 | 5.80 × 10⁷ | 1.79 × 10⁷ | 30.862069 |

### Example 2

### Immunological Characteristics of Hair Follicle Stem Cells

In order to examine the immunological characteristics of the CD34-positive cells isolated according to the above-described isolation method, FACS (fluorescence activated cell sorting) analysis was carried out, and the analysis results are shown in FIG. 7. Antigens analyzed for immunophenotype in this Example showed one or more immunological characteristics selected from among CD34 positivity (B), CD44 positivity (C), CD45 positivity (D), CD133 positivity (E) and CD29 positivity (F). 1×10⁵ cells, identified in the section 3) of Example 1, were washed with a 2% FBS-containing PBS solution and allowed to react with antibodies to each of the antigens at room temperature. Whether the antigens were expressed was analyzed using a flow cytometer.

The analysis results are shown in FIG. 7. As can be seen in FIG. 7, the human hair follicle stem cells of the present invention showed positive responses of more than 90% to CD34(B) that is a typical antibody for mesenchymal stem cells, more than 80% to CD44(C), more than 60% to CD45(D), more than 70% to CD133(E), and more than 90% to CD29(F). In FIG. 7, the control group (A) was the case where the cells were not allowed to react with the antibodies.

### Example 3

### Effects After Administration of Composition Containing Hair Follicle Stem Cells as an Active Ingredient

### (1) Administration of Hair Follicle Stem Cells and Observation in Mice

A syringe containing 1×10⁵ CD34-positive cells transferred therein was placed on ice, and then the cells were administered into nude mice by subcutaneous injection. As a control group, animals without the cells administration were photographed for comparison with the state of mice with the cells administration. The animals used in the test were as follows:
(1) animal species: BALB/cAnNCrjBgi-nu mice
(2) sex and age when purchased: female and 6 week-old
(3) purchased from: Orient Bio Inc., Korea
(4) number of animals purchased: 25 females
(5) inspection and acclimation period: the animals were acclimated in our laboratory for about 1 week, during which general conditions of the animals were observed, and only healthy animals were provided in test.
(6) number of animals used: 20 females
(7) age when tested: 7 week-old females
(8) grouping method: random method
(9) breeding conditions
   Environmental conditions: this test was carried out on an MI rack equipped with a HEPA filter, in a laboratory (room no. 727, building no. 85) in Collage of Veterinary Medicine, Seoul National University, under the following conditions: temperature: 22±3□, relative humidity: 50±10%, number of ventilation: 10-12/hr, illumination time: 12 hr (07:00-19:00), and illumination intensity: 150-200 Lux.
   Breeding cages, breeding density and identification of breeding cages
      During the acclimation and test periods, the animals were bred in polycarbonate MI cages (26×42×18 cm; manufactured by Myoungjin Mechanical Co., Korea) at a density of 5 animals/cage. Each of the breeding cages was labeled with a tag having the test number, animal number and dose written therein.
   Providing feed and drinking water

During the acclimation period, the animals were allowed ad libitum access to solid feed sterilized with high-pressure steam (Purina) and drinking water sterilized with high-pressure steam.

TABLE 2
Grouping of test animals

| | Sex | Number of animals | Animal # |
|---|---|---|---|
| Control group | female | 10 | CF 1~10 |
| Administered group | female | 10 | HSCF 1~10 |

After the hair follicle stem cells of the present invention were administered into the nude mice grouped randomly as described above, the skin of the test animals was observed for 15 days, and the date when hair appeared was recorded. Before the start of the test and at the end of the test, the weight of each of the test animals was measured, and at 16 days after administration (0 day), the test animals were autopsied. For statistical analysis on the test animal's body weight, etc., measured during the test, one-way ANOVA was carried out to examine the significance between the groups, and when the significance was acknowledged, Dunnett's t-test was carried out to examine the statistical significance between the control group and the test group (p<0.05).

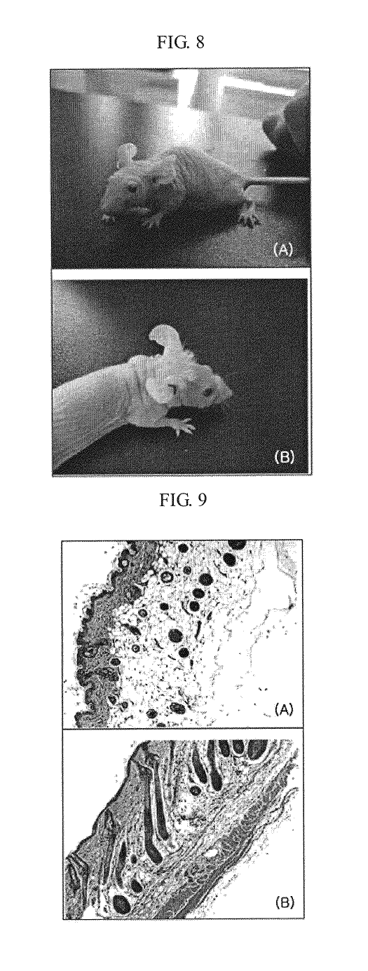

As a result, photographs of the mouse group without the hair follicle stem cell administration (hereinafter, referred to as a “control group”), and the mouse group with the hair follicle stem cell administration (hereinafter, referred to as an “administered group”), are shown in FIG. 8. As can be seen in FIG. 8, it could be observed that, in the nude mice (A) of the control group, no hair appeared (A), whereas, in the nude mice (B) of the administered group, hair appeared mainly on the head portion at 9-12 days after the start of the test.

### (2) H&E Staining of Mouse Scalp Sections

Portions of scalps were removed from the nude mice of each group, fixed in 10% formalin, embedded in paraffin, and sectioned to a thickness of 0.2 μm. The sections were placed on a slide and stained with hematoxylin and eosin to make tissue samples for microscopic examination.

The results of H&E staining of the scalps of the mice of the control group and the test group are shown in FIG. 9. As can be seen in FIG. 9, in the nude mice (A) of the control group, no hair root appeared, whereas, in the administered group, hair roots could be observed in the scalp tissue.

### (3) In Situ Hybridization of Mouse Scalp Sections

In the same manner as in the above H&E staining, portions of scalps were removed from the nude mice of each group, and the scalp tissues of each individual of the animal groups were fixed with a fixing solution consisting of a mixture of 4% paraformaldehyde phosphate solution and 1.5% sucrose solution. The fixed tissues were left to stand at 4□ until they settled in 30% sucrose phosphate solution. Then, each of the tissues was embedded in paraffin and finely sectioned with a tissue microtome by a thickness of 5 μm. Then, the sections were allowed to react with prehybridization solution (50% formamide, 4×SSC, 50 mM DDT, 4× Denhart's solution, X TED, 100 ug/ml of denatured salmon sperm DNA, and 250 μg/ml of yeast RNA) at 42□ for 1 hour. To the resulting solution, DIG-labeled DNA (100 ng/ml) was added and allowed to react with the prehybridization solution for 24 hours, such that a DIG labeled human-specific DNA probe was bound to the mRNA of the nude mouse scalp cells. The tissues were washed two times with each of 2×, 1× and 0.5×SSC solutions for 10 minutes each time, and fixed on a slide glass, followed by drying at room temperature for 2 hours.

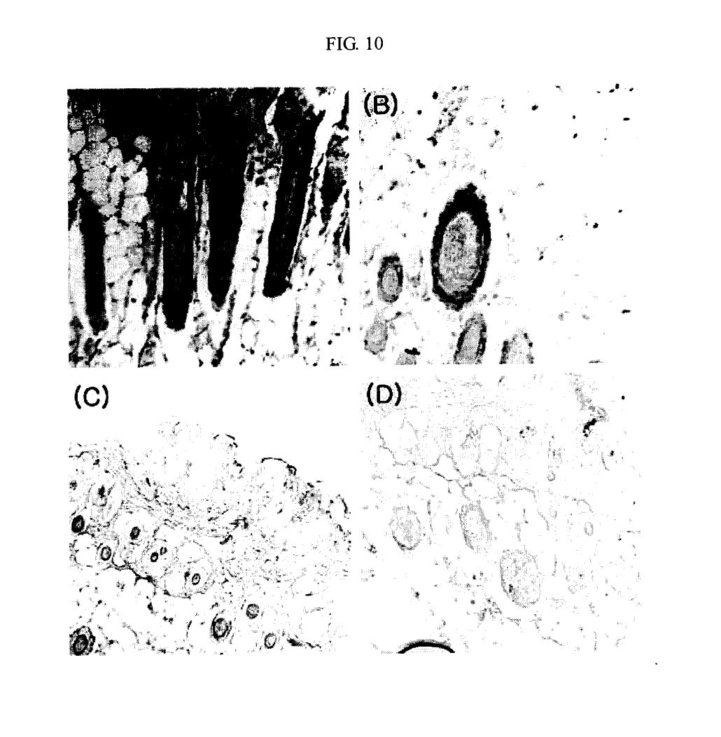

The test results are shown in FIG. 10. As can be seen in FIG. 10, the control group (C and D) was not labeled with the human-specific probe, but the individuals of the administered group were labeled with the human-specific probe around the outer root sheaths (A) and the hair follicles (B).

Based on the prior study results suggesting that stem cells expressing a CD34 cell surface protein are present in the upper regions of the outer root sheath of the mouse hair follicle (the so-called bulge region) (Trempus C. S. et al., J. Invest Dermatol. 120(4):501, 2003), the present inventors extracted scalp tissue directly from a living human body and used the extracted tissue to identify hair follicle stem cells expressing the CD34 cell surface protein. Also, in order to examine the in vivo effects of the hair follicle stem cells, the cells were administered into nude mice by subcutaneous injection, and at 10 days after the administration, hair grown on the head portion of the mice was observed. Moreover, the difference between the tissue having hair grown thereon and the tissue of the control group could be found using the H&E staining method.

Although many studies on hair follicle stem cells are being conducted, there are still no standardized cell culture method and stem cell isolation method. The present invention has suggested the method of culturing scalp cells from hair follicle-containing scalp tissue and the method of identifying hair follicle stem cells from the cells. The culture method can be considered to be a more efficient method compared to the prior art, in that hair follicle cells differentiate into hair follicle stem cells using the method, and the yield of the hair follicle stem cells from the hair follicle cells is about 20-50%. Also, in the present invention, the hair growth effect of the hair follicle stem cells in mice was confirmed.

### INDUSTRIAL APPLICABILITY

As described in detail above, hair follicle-derived stem cells, which are obtained according to the present invention, are classified as autologous adult stem cells, have self-renewal capability, the ability to differentiate into adult hair follicle cells and the ability to induce hair growth, and can be used as a novel cell therapeutic agent against hair loss. In addition, the present invention provides a method for culturing hair follicle cells, which has high yield compared to that of the prior art, as well as a method for identifying hair follicle stem cells.

Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof.

1. A method for isolating hair follicle stem cells, the comprising the steps of:
(a) cutting hair follicle-containing scalp tissue into fine pieces and chemically degrading the cut tissue;
(b) collecting the chemically degraded tissue and culturing the collected tissue in a medium containing 1-30 vol % of serum, in an incubator;
(c) replacing the medium with a serum-free medium, when the tissue adheres to the incubator and then re-culturing the tissue; and
(d) collecting the cultured and proliferated scalp cells from the re-cultured tissue, and isolating hair follicle stem cells showing a positive immunological response to CD34, from the collected cells.

2. The method for isolating hair follicle stem cells according to claim 1, wherein the chemical degradation of step (a) comprises the sequential sub-steps of:
(i) degrading the tissue in a medium, which contains a dispase and a protein complex comprising DNase and protease; and
(ii) degrading the tissue in a collagenase-containing medium.

3. The method for isolating hair follicle stem cells according to claim 1, wherein the medium containing 1-30 vol % of serum in the step (b) is a mixed medium of M199/F12 (a medium obtained by mixing M199 with F12 at a volume ratio of 1:1), added with 0.1-1.0 μg/Ml of insulin, 0.1-1.0 μg/Ml of transferrin, 50-150 units of penicillin, 0.05-0.15 mg/Ml of streptomycin, 0.1-0.5 μl/Ml of neomycin, 1-100 ng/Ml of rEGF (epidermal growth factor), 1-100 ng/Ml of bFGF (basic fibroblast growth factor), 10-200 μg/Ml of normocin, 0.01-0.3 Ml/Ml of fetal bovine serum, and 0.1-10 mM of N-acetyl-L-cystein.

4. The method for isolating hair follicle stem cells according to claim 1, wherein the serum-free medium in the (c) is a serum-free medium containing no normocin.

5. The method for isolating hair follicle stem cells according to claim 1, wherein the step (c) comprises: culturing the tissue in a serum-free medium containing normocin; and then culturing the tissue in the serum-free medium containing no normocin

6. The method for isolating hair follicle stem cells according to claim 5, wherein the serum-free medium containing no normocin is a serum-free keratinocyte medium containing 0.1-10 mM of ascorbic acid.

7. The method for isolating hair follicle stem cells according to claim 5, wherein the serum-free medium containing normocin a mixed medium of M199/F12 (a medium obtained by mixing M199 with F12 at a volume ratio of 1:1), added with 0.1-1.0 μg/Ml of insulin, 0.1-1.0 μg/Ml of transferrin, 50-150 units of penicillin, 0.05-0.15 mg/Ml of streptomycin, 0.1-0.5 μg/Ml of neomycin, 1-100 ng/Ml of rEGF (epidermal growth factor), 1-100 ng/Ml of bFGF (basic fibroblast growth factor), 10-200 μg/Ml of normocin, and 0.1-10 mM of N-acetyl-L-cystein.

8. The method for isolating hair follicle stem cells according to claim 1, wherein said hair follicle stem cells show one or more immunological characteristics selected from the group consisting of CD44 positivity, CD45 positivity, CD133 positivity and CD29 positivity.

9. A composition for the induction of hair growth, which contains the CD34 positive hair follicle stem cells isolated by the method of claim 1, as an active ingredient.

10. A composition for the induction of hair growth, wherein the CD34 positive hair follicle stem cells are administered in an amount of 1×10³ to 1×10¹² cells.

11. A composition for induction of hair growth, comprising CD34 positive hair follicle stem cells.
